(19) United States
(12) Patent Application Publication
(10) Pub. No.: US 2020/0345534 A1
(43) Pub. Date: Nov. 5, 2020

(54) DENTAL APPLIANCES, DENTAL SOLUTION SELECTION, AND ENHANCEMENTS AND SYSTEMS AND METHODS OF PRODUCING THE SAME

(71) Applicant: Kelly LUCAS, Wasilla, AK (US)

(72) Inventor: Kelly LUCAS, Wasilla, AK (US)

(21) Appl. No.: 16/643,042

(22) PCT Filed: Aug. 31, 2018
(86) PCT No.: PCT/US2018/049090

§ 371 Date: Feb. 28, 2020

Related U.S. Application Data
(60) Provisional application No. 62/552,634, filed on Aug. 31, 2017.

(51) Int. Cl. A61F 5/56; A61C 7/00; A61C 7/08; A61C 7/10; A61C 7/36; G05B 19/4097

(52) U.S. Cl. CPC A61F 5/566; A61C 7/002; A61C 7/08; A61C 7/10; G05B 2219/35012; G05B 19/4097; A61C 2204/00; A61F 2005/563; A61C 7/36

(57) ABSTRACT

A method of producing a retention piece having a therapeutic package configured to provide a desired therapeutic profile for the recipient, the method including: obtaining data descriptive of mandibular and maxillary arches of the recipient; specifying an appropriate guidance package based on the desired therapeutic profile for the recipient; modeling a virtual representation of at least one retention piece configured to fit at least one of the mandibular and maxillary arches; and generating data that can be used by a computer aided machining (CAM) process to create at least one physical retention piece comprising the specified therapeutic package configured to provide the desired therapeutic profile.

### CROSS-REFERENCE TO RELATED APPLICATION

This Application claims priority under 35 U.S.C. § 119(e) to U.S. Provisional Patent Application No. 62/552,634, filed Aug. 31, 2017, the contents of which is hereby incorporated in its entirety as if fully set forth herein.

### TECHNICAL FIELD

The various embodiments of the present disclosure relate generally to oral retention pieces, enhancements thereto, selection of a dental solution, and systems and methods for producing the same.

### BACKGROUND

Dentists are often faced with the problem of trying to correct numerous issues with the jaw and teeth. For example, correcting movement of a patient's mandibular arch with respect to their maxillary arch to solve a number of problems, such as bruxism, sleep apnea, TMD (Temporo-mandibular dysfunction), and full mouth reconstruction has traditionally been difficult. Traditionally, dental professionals have constructed customized appliances that are typically attached to the maxillary and/or mandibular teeth. Also, dentists construct one or two arch prosthetic reconstructions. The customized appliances and/or reconstructions allow the dental professional to consider a patient's particular malocclusion and other factors to place the patient's jaw in centric relation or another predetermined index position. While helpful, these customized appliances and reconstructions typically require substantial time and effort to create and modify for each patient. Existing appliances and reconstructions may also be limited for patients with missing or periodontally weakened teeth or specific malocclusions, and only provide limited anterior guidance. Furthermore, related art products and approaches are dependent upon individual practitioners and individual labs for creating appliances and restorations for a patient.

The inventor of the embodiments described herein has invented numerous oral retentive pieces, guidance packages, dental solutions, and systems for making and using the same to provide a number of improvements to the art, which are described in, for example, U.S. patent application Ser. Nos. 13/573,283, 13/774,033, 13/774,920, 13/918,754, 14/083,467, and 14/748,805, and PCT/US2017/049545, the disclosures of which are incorporated herein by reference in there entireties as if fully set forth below.

Additional aspects of oral retentive pieces, guidance packages, dental solutions, and systems for making and using the same not considered in the prior art may provide additional benefits.

### SUMMARY

The present disclosure relates oral retention pieces, enhancements thereto, selection of a dental solution, and systems and method for producing the same. According to some embodiments, there is provided a method of producing a retention piece having a therapeutic package configured to provide a desired therapeutic profile for the recipient, the method including: obtaining data descriptive of mandibular and maxillary arches of the recipient; specifying an appropriate guidance package based on the desired therapeutic profile for the recipient; modeling a virtual representation of at least one retention piece configured to fit at least one of the mandibular and maxillary arches; and generating data that can be used by a computer aided machining (CAM) process to create at least one physical retention piece comprising the specified therapeutic package configured to provide the desired therapeutic profile.

The modeling the virtual representation of at least one retention piece may include forming, within the virtual representation, one or more negative spaces.

The one or more negative spaces may be configured to produce a more shock absorbing area of the at least one retention piece.

The method may further include creating the at least one physical retention piece based on the generated data, the at least one physical retention piece including, within the negative space, at least one from among an electrolytic gel, one or more sensors, a tracking device, a computer, and a microgenerator.

The method may further include creating the at least one physical retention piece based on the generated data, the at least one physical retention piece including, within the negative space, an intermediary implant attachment mechanism that will accept an implant.

The modeling the virtual representation of at least one retention piece may include forming, within the virtual representation, a plurality of negative spaces connected by channels within the virtual representation.

The modeling the virtual representation of at least one retention piece may include forming, within the virtual representation, a plurality of attachment points on an external surface of the at least one retention piece.

The method may further include creating the at least one physical retention piece based on the generated data, the at least one physical retention piece including, connected to the plurality of attachment points, at least one from among an elastic band, a guidance rod, a spacer, a spring, a flange, a magnet, a worm gear, a jackscrew, a piston, and an expansion screw.

The therapeutic package may include palatal suture manipulation, and the at least one retention piece comprises a rapid palatal expander.

The method may further include modeling a plurality of virtual representations of at least one retention piece configured to fit at least one of the mandibular and maxillary arches and simulating a change of the mandibular and maxillary arches based on the plurality of virtual representations.

The at least one retention piece may include at least one from among: one or more modifiers to adjust a maximum closure position of a sleep apnea appliance, a two-way expander, a three-way expander, twin block appliance features, and features of interacting inclined planes for myofunctional manipulation.

The modeling the virtual representation of the at least one retention piece may include modeling the virtual representation based on at least one mathematical principle of aesthetics.

The at least one mathematical principles of aesthetics may include tooth size and shape in a continuum of parameterization. The at least one mathematical principles of aesthetics may include the use of golden proportions of tooth size and shape.

Modeling the virtual representation of the at least one retention piece may include modeling the virtual representation to overlay the at least one of the mandibular and maxillary arches with a golden proportion of tooth size and shape.

Modeling the virtual representation of the at least one retention piece may include modeling the virtual representation with variation in the golden proportion to provide symmetry of teeth.

Modeling the virtual representation of the at least one retention piece may include modeling a manufactured surface on at least a portion of the at least one retention piece. The manufactured surface may include at least one of a rough surface, a textured surface, and a meshed surface.

The method may further include: creating the at least one physical retention piece based on the generated data; and performing a surface treatment to the manufactured surface.

According to some embodiments, there is provided a system for producing a retention piece having a therapeutic package configured to provide a desired therapeutic profile for the recipient, the system including: at least one processor; and at least one memory having stored thereon computer program code that, when executed by the at least one processor, controls the processor to: obtain data descriptive of mandibular and maxillary arches of the recipient; specify an appropriate guidance package based on the desired therapeutic profile for the recipient; model a virtual representation of at least one retention piece configured to fit at least one of the mandibular and maxillary arches; and generate data for a computer aided machining (CAM) process to create at least one physical retention piece comprising the specified therapeutic package configured to provide the desired therapeutic profile.

The modeling the virtual representation of at least one retention piece may include forming, within the virtual representation, one or more negative spaces.

The one or more negative spaces may be configured to produce a more shock absorbing area of the at least one retention piece.

The computer program code, when executed by the at least one processor, may further control the processor to output instructions to create the at least one physical retention piece based on the generated data, the at least one physical retention piece including, within the negative space, at least one from among an electrolytic gel, one or more sensors, a tracking device, a computer, and a microgenerator.

The computer program code, when executed by the at least one processor, may further control the processor output instructions to create the at least one physical retention piece based on the generated data, the at least one physical retention piece including, within the negative space, an intermediary implant attachment mechanism that will accept an implant.

The modeling the virtual representation of at least one retention piece may include forming, within the virtual representation, a plurality of negative spaces connected by channels within the virtual representation.

The modeling the virtual representation of at least one retention piece may include forming, within the virtual representation, a plurality of attachment points on an external surface of the at least one retention piece.

The computer program code, when executed by the at least one processor, may further control the processor output instructions to create the at least one physical retention piece based on the generated data, the at least one physical retention piece including, connected to the plurality of attachment points, at least one from among an elastic band, a guidance rod, a spacer, a spring, a flange, a magnet, a worm gear, a jackscrew, a piston, and an expansion screw.

The at least one retention piece may include at least one from among: one or more modifiers to adjust a maximum closure position of a sleep apnea appliance, a two-way expander, a three-way expander, twin block appliance features, and features of interacting inclined planes for myofunctional manipulation.

### BRIEF DESCRIPTION OF THE DRAWINGS

The following detailed description is better understood when read in conjunction with the appended drawings. For the purposes of illustration, there is shown in the drawings example embodiments, but the subject matter is not limited to the specific elements and instrumentalities disclosed.

FIG. 1 illustrates examples of golden proportions in dentistry.

FIGS. 2 and 3 illustrate guidance package solutions with attachment points, in accordance with certain example embodiments.

FIGS. 4A and 4B illustrate sectioned maxillary guidance package solutions with internal attachment points, in accordance with certain example embodiments.

FIG. 4C illustrates a twin block appliance in accordance with certain aspects of the present disclosure.

FIG. 4D illustrates a guidance package solution appliance with attachment points for interlocking flanges, in accordance with certain example embodiments.

FIG. 4E illustrates a sectioned appliance with attachment points for interlocking flanges, in accordance with certain example embodiments.

FIGS. 5A-5D illustrate examples of a CAD/CAM production including a surface for treated or manipulated with subsequent processes on appliances and restorations, in accordance with certain example embodiments.

FIG. 6 illustrates dental appliances with negative spaces including created pathways according to certain example embodiments.

FIG. 7 illustrates negative or empty spaces in appliances and restorations, in accordance with certain example embodiments.

FIGS. 8A and 8B illustrate negative spaces, platforms, and attachment points according to certain example embodiments.

FIG. 9 is a block diagram of an example computer system capable of implementing certain aspects of the present disclosure.

### DETAILED DESCRIPTION

To facilitate an understanding of the principles and features of the present invention, various illustrative embodiments are explained below. To simplify and clarify explanation, the invention is described below as applied to oral retention pieces comprising guidance packages for obtaining a desired movement profile for a patient's mandibular and maxillary arches.

The components, steps, and materials described hereinafter as making up various elements of the invention are intended to be illustrative and not restrictive. Many suitable components, steps, and materials that would perform the same or similar functions as the components, steps, and materials described herein are intended to be embraced within the scope of the invention. Such other components, steps, and materials not described herein can include, but are not limited to, similar components or steps that are developed after development of the invention.

Varying dental solutions may be created for a given dental patient to solve certain dental problems. For example, a solution may be generated based on user-specific data (e.g., mandibular and maxillary arch information, a desired index position, and temporomandibular joint information). These solutions may include providing a guidance package to a virtual articulator, and creating one or more virtual dental solutions based on the guidance package. The virtual dental solutions may then be produced using, for example, CAD-CAM or some other manufacturing technique. In many circumstance, a plurality of dental solutions may be available for a given patient based on a given guidance package. This may include solutions of a similar type with differing shapes or shapes formed over a continuum, or of different types (e.g., appliances, restorations, and restorative/surgical/implant placement guides). Within this continuum, anterior protrusive guidance may be located on the anterior aspect of the appliance, or on the posterior lateral aspects of the appliance. These virtually planned final shapes may look very different from one another and from the personalized canonical guidance and stop profile from which they were derived but will provide the exact same guidance and stop profile that was determined to be therapeutic by the operator.

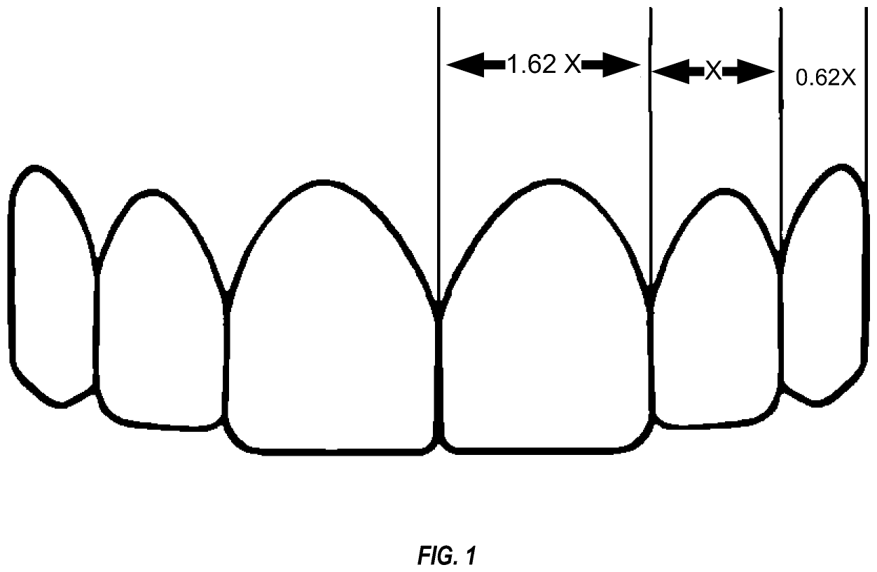

In some embodiments, generating or selecting one or more dental solutions for a given dental patient that includes one or more false teeth may include aesthetic considerations. In other words, it may be desirable to consider aesthetics when selecting or discarding potential solutions. In some cases, for example, a clinician and/or patient may choose to apply “golden proportions” or other aesthetic criterion for the final product selection. In some circumstances, the system may take into account aesthetics when generating (e.g., parameterizing) particular dental solutions. These aesthetic considerations may influence all or a portion of the guidance package. As will be understood by one skilled in the art, “golden proportions” describe certain smile design principles when initiating the restoration of teeth in the anterior region of the mouth, for example, as illustrated in FIG. 1. Also, mathematical principles of tooth size and shape may be incorporated into the guidance package solution for additional criterion other that aesthetics. As an example, the absolute sizes of teeth will be different when contrasting a smaller dental arch verses and larger dental arch. Using mathematical principles of comparative tooth size of each tooth within that arch can be used to provide the correct size “teeth” in a given guidance package solution regardless the arch size.

In some cases, preferred position of the incisal edges, incisal plane, midline position, the length and width of the teeth, tissue height, axial inclination, embrasure form, line angles, and surface texture will be considered. The concept known as the Golden Proportion provides mathematical guidelines for tooth preparation and fabrication of the restorations. Application of this principle when designing dental solutions will, therefore, help to improve design, functional results and patient satisfaction.

The Golden Proportion is based on a mathematical proportion that is acquired from mathematics and nature. The Golden Proportion is one where the ratio of the shorter segment to the longer segment (x:1−x) is equal to the ratio of the longer segment to the whole segment (1−x:1). The mathematical equation that is solved for x is: x/(1−x)=(1−x)/1. Solving this equation results in a shorter segment length of approximately 0.382 and the longer segment length of approximately 0.618. This is the only ratio that solves the equation. Using the Golden Proportion in the development of size and shape relationships for teeth in a guidance solution may be a consideration. In some implementations, a system may consider these principles in determining the width of the teeth as they relate to each other in a dental solution. There are many important design considerations that accompany application of the Golden Proportion. For example, considerations may include determining the incisal edge position, incisal plane, gingival plane, and/or central incisor length, prior to applying the Golden Proportion. As applied to the maxillary teeth, the Golden Proportion requires a 62% reduction in the viewing width of each tooth, beginning with the central incisor, and proceeding posteriorly. Further, in some cases, tooth size and shape may be considered as part of aesthetic planning, but may also provide the correct ratios of tooth size when considering ratios of incisors, canines, premolars and molars to provide these correct proportions for a specific arch size and shape.

As an example application of the Golden Proportion principals, a central incisor will have a viewing width approximately 1.62 times (1.62X) the viewing width (X) of a lateral incisor, the lateral incisor's viewing width (X) will be approximately 1.62 times the viewing width of the viewing width of the cuspid (0.62X), which will have viewing width 1.62 times the viewing width of the first premolar, and which will have a viewing width 1.62 times the viewing width of the second premolar. This allows for a smile dominated by the central incisors, with the other teeth becoming progressively smaller.

In some cases, a variation of the Golden Proportion may be considered to develop symmetry, dominance, and proportion for aesthetically pleasing smiles. Simplifying these proportions, the assigned width of the six anterior teeth is a percentage of the total viewing width of the six anterior teeth: central incisor 25%, lateral incisor 16%, and cuspid 9%. These percentages illustrate the dominance of the central incisors, which are 50% of the cuspid-to-cuspid viewing width.

This is merely an example implementation, and a number of different aesthetic criterion (including the golden proportions or other aesthetic criterion) may be applied when designing a guidance package solution for a specific patient according to some implementations. These various aesthetic criteria may be applied to maxillary anterior teeth, mandibular anterior teeth, or any other subset of prosthetic teeth or modified natural teeth as part of the guidance package solution. Through guidance package systems according to the present disclosure, using standardized solution platforms programmed application of mathematic aesthetic principles are possible. In contrast, in the related art, human operator planning is required. In some cases, dental solution generation or selection may consider attachment points to the external aspects of appliances, restorations, and surgical/restorative/implant placement guides for further utility of the product. For example, if a dental solution is generated virtually, platforms and/or attachment points may be designed for other technologies, such as, as non-limiting examples, sensors (e.g., force sensors), or to provide other exploitation or manipulation of the solution. Accordingly, the utility of the dental solution could be enhanced over that found in the related art.

The guidance system may provide preprogrammed solutions according to one or more considerations. As a non-limiting example, orthodontic/orthopedic solutions may be configured to simultaneously address TMJ health and function. Certain guidance package standardized solutions may incorporate orthopedic, orthodontic, TMD and/or TMJ considerations into a hybrid solution. As a non-limiting example, a dental solution may include a hybrid orthodontic/guidance package appliance configured to address, e.g., a severe class 2 malocclusion. The hybrid orthodontic/guidance package two-piece appliance could feature attachment points for springs, elastics, or another force system to aid in the transverse movement of tissues planned for the particular patient. In some cases, there may be provided a preprogrammed application of these platforms and attachment points. The guidance package system may provide standardized solution platforms, which enables the preprogramming of platforms and attachment points. Related art solutions rely on human operator planning. Meanwhile, according to certain aspects of the present disclosure, for the first time, standardized solutions using the guidance package system can include standardized criterion for the placement of these platforms and/or attachment points for a specific aim or solution and may be assigned to a certain standardized guidance package solution.

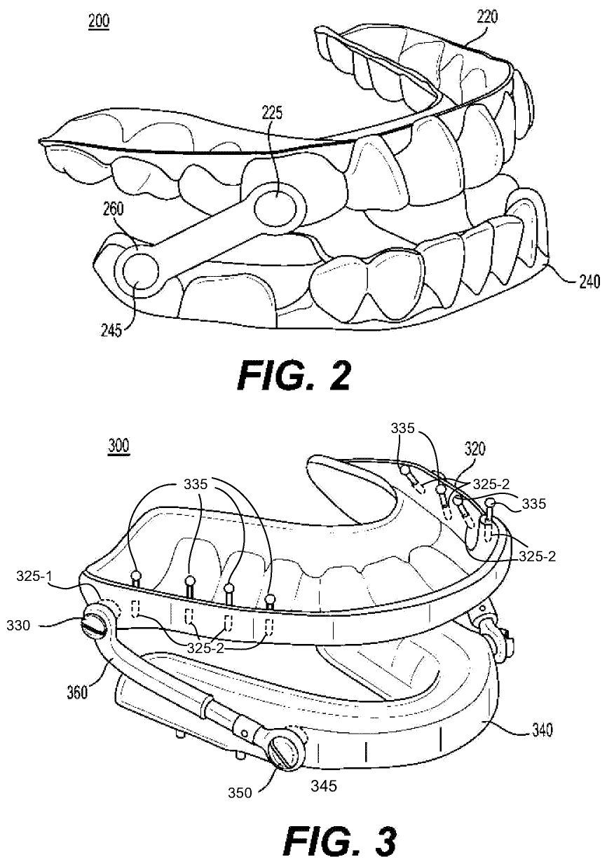

FIG. 2 illustrates a two-piece guidance package derived appliance 200 including attachment points. As illustrated in FIG. 2, the guidance package derived appliance 200 includes maxillary retentive piece 220 and mandibular retentive piece 240. Attachment points 225 and 245 are formed, respectively, on maxillary and mandibular retentive pieces 220 and 240. For example, attachment points 225 and 245 may be formed as raised knobs on a side surface of maxillary retentive piece 220 and mandibular retentive piece 240. Elastics 260 are attached to the attachment points 225 and 245 to provide a tension force to aid in the reduction of a skeletal class 2 malocclusion.

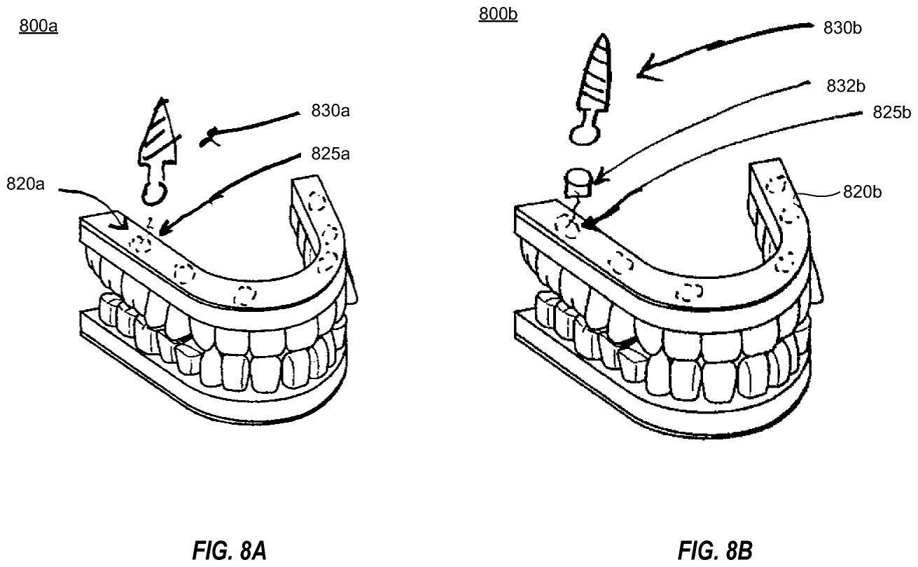

FIG. 3 illustrates another two-piece guidance package derived appliance 300 that includes attachment points and negative spaces. As illustrated in FIG. 3, the guidance package derived appliance 300 includes maxillary retentive piece 320 and mandibular retentive piece 340. Attachment points 325-1 and 345 are formed, respectively, on maxillary and mandibular retentive pieces 320 and 340. For example, in some cases, attachment points 325-1 and 345 may be voids for screws 330 and 350 embedded in a side surface of maxillary retentive piece 320 and mandibular retentive piece 340. In some cases, maxillary retentive piece 320 and mandibular retentive piece 340 may be formed with voids configured to receive attachment points 325-1 and 345 (see, e.g., FIGS. 8A and 8B). Piston 360 is attached to the screws to provide a tension force to aid in the reduction of a skeletal class 3 malocclusion. Piston 360 may act as a shock absorber that holds the jaw in a protrusive orientation. In some cases, piston 360 can have dampening or shock absorption as the mandible functions and delivers these forces more gently. Negative spaces 325-2 may be used to add additional retentive capability of a retentive piece. A ball clasp 335 or other retentive feature may be placed into these negative spaces 325-2. The negative spaces 325-2 may be configured to directly accept the retentive feature 335 and/or to accept a housing that will in turn accept the retentive feature 335. In some cases, a housing may be used to increase an adjustable nature of a retentive 335 or other feature.

One of ordinary skill will understand that the attachment points, attachments, and negative spaces illustrated in FIGS. 2 and 3 are merely examples. One of ordinary skill will understand in light of the present disclosure that attachment points, voids, and or platforms may be formed and configured to accept one or more of elastics, pistons, springs, expansion screws, worm gears, jackscrews, flanges, and other force systems. Based on the relative orientation and positioning of attachment points and the type of attachment utilized, the attachments may help lateral development, vertical development, and/or anterior-posterior development within the patient's stomatognathic system. In some cases, the attachment points may be placed along a continuum of spots in accordance with a desired tension orientation. In some cases, the system may provide a plurality of options that meet the given requirements. In some cases, the system may designate one or more attachment points or potential attachment points based on an analysis of user specific data. For example, the system may parameterize a dental solution that include attachment points based on the patients TMJ data.

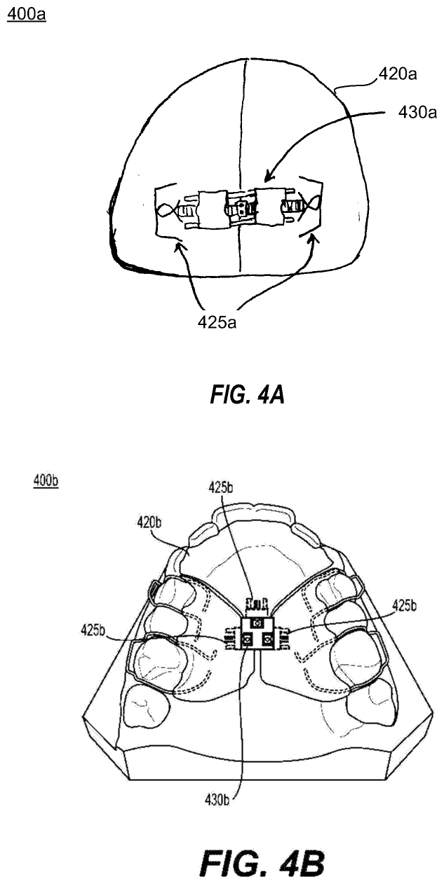

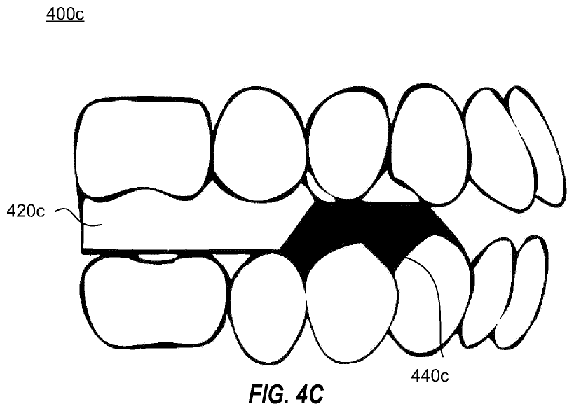

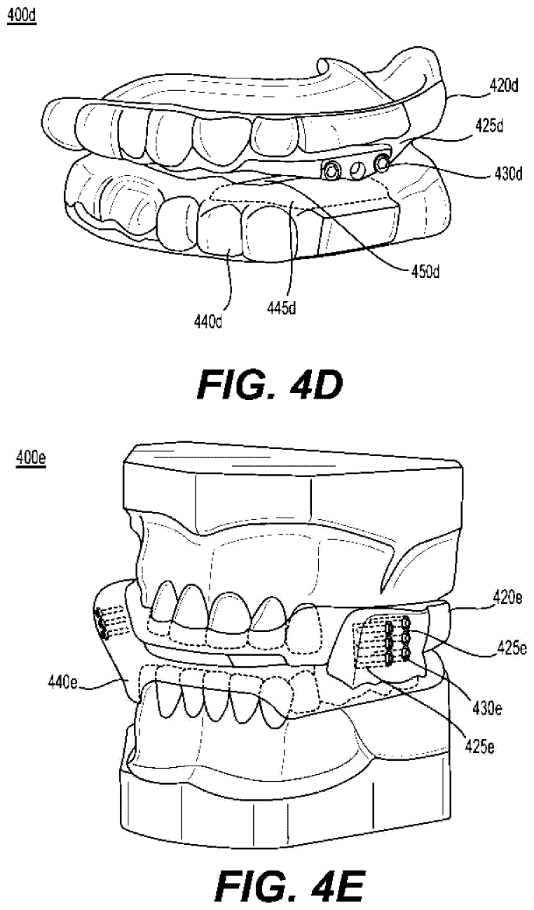

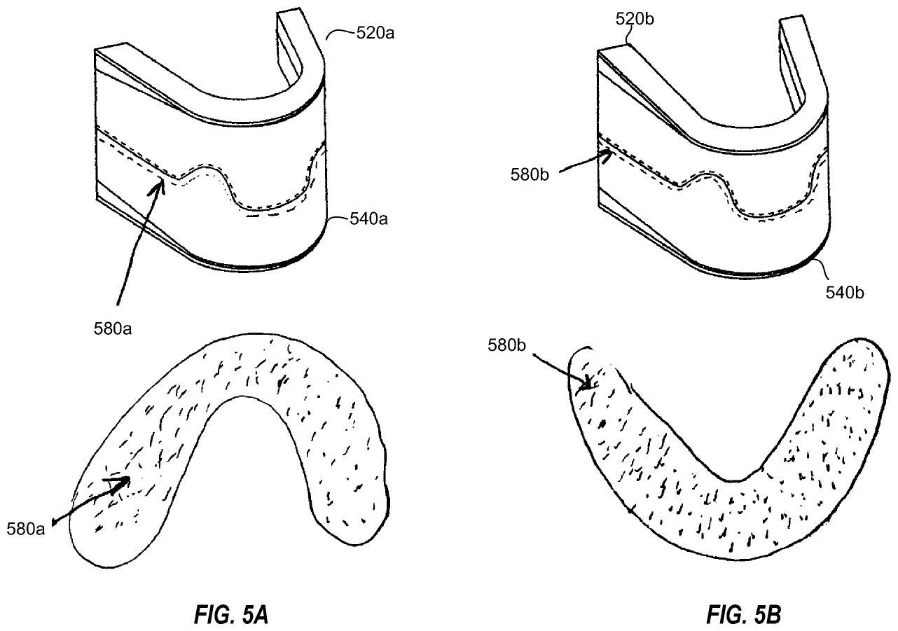

FIG. 4A illustrates a palatal view of a sectioned maxillary guidance package solution 400a with attachment points for an expansion screw according to an example embodiment. As illustrated in FIG. 4A, the guidance package solution 400a includes maxillary retentive piece 420a with attachment points 425a formed therein. Expansion screw 430a may be attached to attachment points 425a and may be configured to provide an expansive force to aid in the reduction of a bilateral posterior crossbite in an adolescent by expansion of the maxillary arch. In some cases, the maxillary guidance package derived appliance may be CAD/CAM planned and constructed to be split down the midsagittal plane to allow the expansive force of the expansion screw to manipulate the Palatal suture. This enables expansion of the maxillary arch and therefore posterior crossbite correction and improvement of the airway providing an effect similar to an RPE (rapid palatal expander), but within a guidance package solution. In some cases, attachment points 425a may include voids configured to receive expansion screw 430a. In some cases, a hinge may replace expansion screw 430a within attachment points 425a. By expanding the maxillary arch with the hinge in this mid sagittal position, the maxillary arch may be opened in a way that tips the maxillary molars facially. These are merely examples, and one of ordinary skill will recognize that various dental solutions (e.g., guidance package solutions) may be designed and developed in segments for various purposes. For example, tension, compressive or expansive force may be applied between the sections to effect change within the structure or orientation of a patient's dental system.

FIG. 4B illustrates a guidance package potential hybridization though development and incorporation of a three-way expander. In FIG. 4B, the guidance package solution 400b includes maxillary retentive piece 420b with attachment points 425b formed therein. A three-way expander 430b may be attached to attachment points 425b and may be configured to provide an expansive force to aid in arch expansion. For example, three-way expander 430b may include two jack screws, one in the midsagittal plane for midsagittal plane expansion, and another to expand anteriorly the premaxilla segment for overall maxillary arch expansion.

FIG. 4C illustrates a guidance package solution that may include features of a twin block appliance. In a child or growing adolescent, inclined planes placed in certain strategic positions and planes that may interact as the mouth closes, opens and functions, restriction of one arch development can be achieved while simultaneously encouraging development of the other arch. These inclined planes may interact as the patients' mandible functions in relation to the maxilla. As an example, these forces may be applied in a way that myofunctional forces will act to reduce a Class 2 malocclusion. Hybrid guidance package/twin block appliances may be constructed for full-time wear. They may achieve rapid functional correction of malocclusion by the transmission of favorable occlusal forces to occlusal inclined planes that cover the posterior teeth. Referring to FIG. 4C, guidance package/twin block appliance 400c may with a variety of components such as expansion screws, springs and clasps may be included in a guidance package solution for a variety of treatment goals. In some cases, these guidance package derived appliances may have more than one template of treatment philosophy from which to be derived and may have other planes of action for correction or development. These hybrid appliances may include advantages from the guidance package system of standardization and calculations based on different variables to include patient specific TMJ data and may include principles of twin block myofunctional appliances such as arch development. One of ordinary skill will recognize that these are merely examples, and that various dental solutions (e.g., guidance package solutions) may be designed and developed in segments for various purposes. For example, tension or expansive force may be applied between the sections to effect change within the structure or orientation of a patient's dental system.

Platforms and attachment points may be included on any location on any guidance package solution to apply forces in any plane on one arch or intra arch forces in any plane to enhance utility of a guidance package solution. These attachment points/platforms/negative spaces may be configured to allow auxiliaries in any plane to include transverse plane movement and vertical movement to include extrusive and intrusive forces. These forces may be applied, tension, and/or spring forces provided by all categories of force producing systems. Examples of force systems could include elastics, pistons, wire, spindles, worm drive, expansion or retraction screw, nickel titanium wire, and all other traditional and non-traditional orthodontic force systems and auxiliary orthodontic force systems.

These attachment points may be used to enhance utility of the guidance package system, the utility of orthodontic, orthopedic and hybrid guidance package systems. In some cases, voids, attachment points, and platforms may be incorporated into a guidance package solution to enhance a sleep apnea appliance. For example, a sleep apnea appliance may be planned to provide an exact new maximum closure position (e.g., a relative positioning of a user's mandible and maxilla when the mouth is fully closed as guided by the guidance package solution). In other cases, the guidance package system could include voids, attachment points and/or platforms for one or more of worm gears, elastics, interlocking flanges or other modifiers so that the end user may, within a defined range, fine tune the new maximum closure position to be maximally therapeutic for sleep apnea in the vertical, protrusive, and lateral mandibular positioning. For example, if a patient were to lose weight after a sleep apnea appliance were made, the exact position of maximum therapeutic effectiveness for sleep apnea may change. By incorporating these modifiers (or attachment points, voids, or platforms to add modifiers) in the original design, the appliance may be kept in service for the patient for an extended period of time as the user's condition changes. Certain aspects of the present disclosure make possible standardized modifiers placed in standardized locations, for example, by providing standardized medical and dental solutions for stomatognathic system that may be individualized for a specific patient. In some cases, voids to directly accept modifiers like interlocking flanges. These voids may be designed to directly accept interlocking flanges into both retentive pieces of a guidance package solution or accept housings that will then accept the interlocking flanges.

FIG. 4D illustrates a guidance package system 400d configured to accept interlocking flanges in the anterior aspect of a sleep apnea appliance. Referring to FIG. 4D, guidance package system 400d maxillary retentive piece 420d with attachment point 425d (e.g., void 425d) formed therein. Flange 430d may be attached attachment points 425d (e.g., inserted into void 425d). Similarly, mandibular retentive piece 440d includes attachment point 445d, and flange 450d may be attached thereto. This flange may include an adjustment mechanism that allows a user to adjust airway to be optimally therapeutic for sleep apnea. FIG. 4E illustrated an apparatus 400e with voids 425e preprogrammed in the lateral aspects of the maxillary and mandibular retentive pieces 420e and 440e to accept interlocking flanges 430e or housings for them. One of ordinary skill will recognize, in view of the present disclosure, additional and/or alternative laterally located preprogrammed negative spaces could be configured to receive, as non-limiting examples, expansion and retraction screws, springs, or other force systems. In light of the present disclosure, one of ordinary skill will recognize that the parameterizations of choice of a guidance package system solution may include inclined planes in either anterior or posterior lateral areas. The guidance system could be preprogrammed to include these voids for interlocking flanges based on the choice of parameterization.

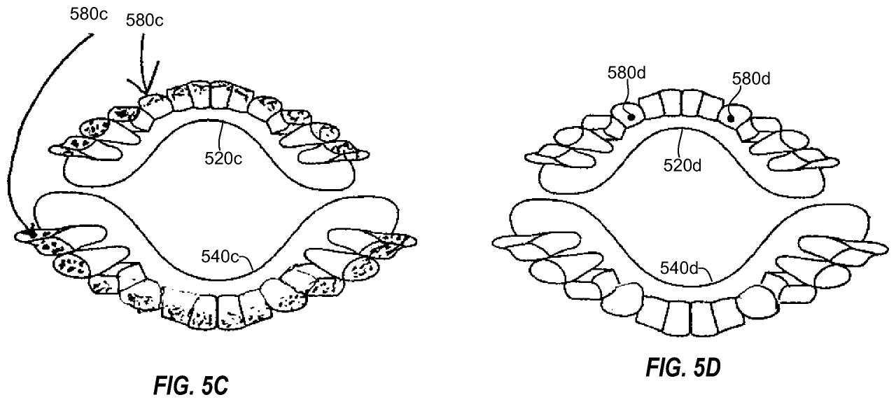

FIGS. 5A-5D illustrate manufactured surfaces of various appliances according to certain embodiments. Referring to FIGS. 5A-5D, maxillary and mandibular retentive pieces 520a-d and 540a-d include manufactured surfaces 580a-d. For example, certain materials are not conducive for CAD/CAM manufacturing. Accordingly, such surface treatments may not be easily applied. Therefore, in some cases, printed surfaces may be designed to facilitate post-CAD/CAM processing. For example, surfaces 580a-d may be, as non-limiting examples, “rough” (e.g., include intentional small variations), textured, meshed, such that magnetic coatings may be applied to attract or repel certain surfaces of the guidance package solution to encourage or discourage certain movements and jaw positions. Surface coatings could include, as non-limiting examples, harder surfaces, softer surfaces, adhesive surfaces, a chemical, a medicament, an anesthetic, a medium to influence the patient at a molecular level, a cellular level, and/or at a tissue level, an electrical medium, a communications medium, a spongy coating, an antimicrobial coating, or a medication releasing medium. In view of the present disclosure, one of ordinary skill will recognize that these surfaces may be located anywhere on the retentive piece to include the functional side and the tissue side.

The surface or internal space may be CAD/CAM prepared to receive a mesh sensor or a mesh sensor created within or on the appliance or restoration by the CAD/CAM process.

In some implementations, potential voids, platforms, and attachment points that could be incorporated into a standardized guidance package solution that could then be used to modify the functionality of a sleep apnea appliance.

In some examples, negative or empty space may be planned, and CAD/CAM produced to allow components to be placed to enhance strength or provide features to the guidance package solution that may not be conveniently produced using CAD/CAM. One example would be to create an empty space in which to place a reinforcing member to increase the strength of the appliance. As an example, it may not be convenient to utilize a 3D printer that could produce both plastic and steel components. Instead a negative space may be designed within the appliance and planned as part of the guidance package solution. Later, a reinforcing member may be placed before delivery of the appliance or restoration to the patient.

Alternatively, negative or empty spaces may be used to make areas less rigid. An operator may create an empty space to subsequently place a substance (such as a gel or some other material with a certain viscosity or property) not conveniently added with CAD/CAM production. As an example, a negative space could be created to produce a more shock absorbing area of an appliance. As another example, a gel could be placed into a negative space to serve as an electrolyte, and/or as part of a larger sensor or control system.

In another application, a computer chip associated with sensors and other devices may be inserted into the negative space to respond to certain activities, situations, and behaviors. As an example, a negative space could be designed to receive a computer chip connected to a pressure sensor to detect bruxism. When bruxism is detected, the computer chip may activate a device such as a vibrator to modify behavior.

Another use of negative spaces would be for placement of a tracking device in order to track a location of the appliance or restoration.

As another example. a microgenerator may be placed in a negative space, which may generate electricity using kinetic energy or compressive force of the jaw. The electricity may be stored in a rechargeable battery (located in the same or other negative space), and may be used to provide power to other devices in the solution (such as vibrators, locator devices, computer system and/or any other device that may be included for therapeutic or other purpose). The negative or empty space could be used to place a switch that may be activated on purpose by the patient or activated by certain movements, behaviors, or set of conditions.

In another example, a certain surface of the guidance package system may have a void or hole included that could then be subsequently filled or treated to change the characteristics of the guidance package solution.

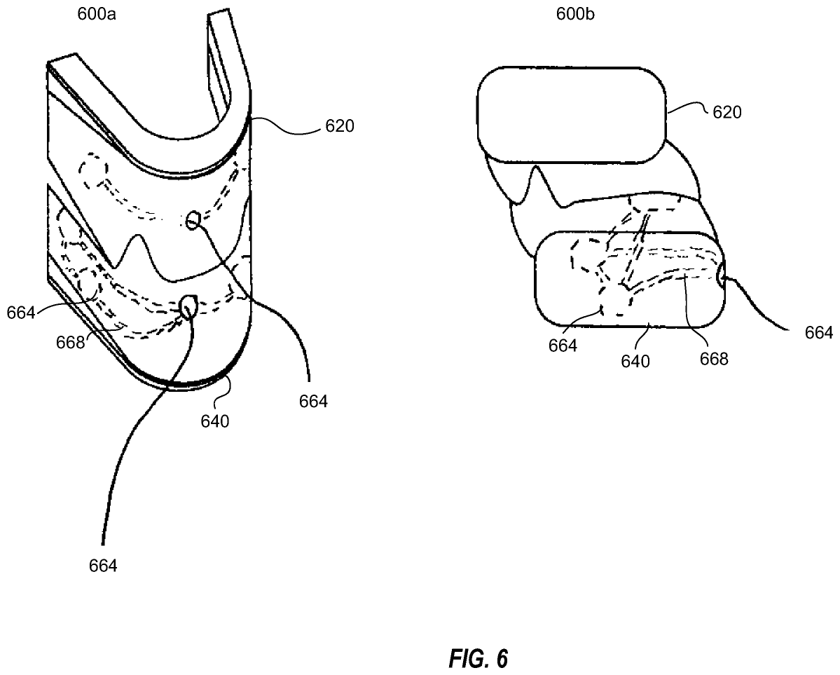

In some cases, a negative or empty space may be designed within a dental solution (e.g., an appliance). This space may be provided for force or other sensors to measure any number of human mouth variables. One example would be sensors placed in negative spaces that measure different bruxism forces on a particular patient. In some cases, the dental solution may be formed around one or more sensors (e.g., using computer-aided manufacturing to build the solution around a sensor). In some cases, parameters and locations of a particular guidance solution or various guidance solutions could be changed to measure forces or other criteria relative to these changes of guidance location. Sensors may be placed to measure any variable of the human mouth. Examples include temperature, moisture, sound sensors (e.g., to record clicking and/or popping of the TMJ(s)), sensors for microbial studies (e.g., to analyze or quantify bacteria that cause tooth decay), chemical studies (e.g., to analyze or quantify chemicals that cause tooth erosion), excretion studies (e.g., to measure specific glandular excretion rates, times and quantities), movement recorders (e.g., to study activity of a particular patient's jaw or tongue or other movement of interest). A variety of sensors may provide a platform for complex studies (e.g., movement sensors and sound sensors to correlate certain movements with TMD clicks and pops). These sensors may be stand alone or may be connected or connectable to external power or monitors through wired or wireless connections. Pathways for wires or other communications or power transmission may be CAD/CAM planned by the guidance package system, e.g., as shown in FIG. 6. In some cases, a researcher may utilize a system within the present disclosure to create a series of dental solutions with negative spaces for one or more sensors.

FIG. 6 illustrates dental appliances 600a and 600b with negative spaces including CAD/CAM created pathways according to certain example embodiments. Referring to FIG. 6, dental appliances 600a and 600b include maxillary and mandibular retentive pieces 620 and 640. Formed therein are voids 664. Pathways 668 are formed between the voids 664, to allow communication (e.g., electrical communication or power lines) therebetween. In some cases, the voids 664 may include one or more of contact points for an external monitor (e.g., a connection port), a wireless communicator, and/or a power supply.

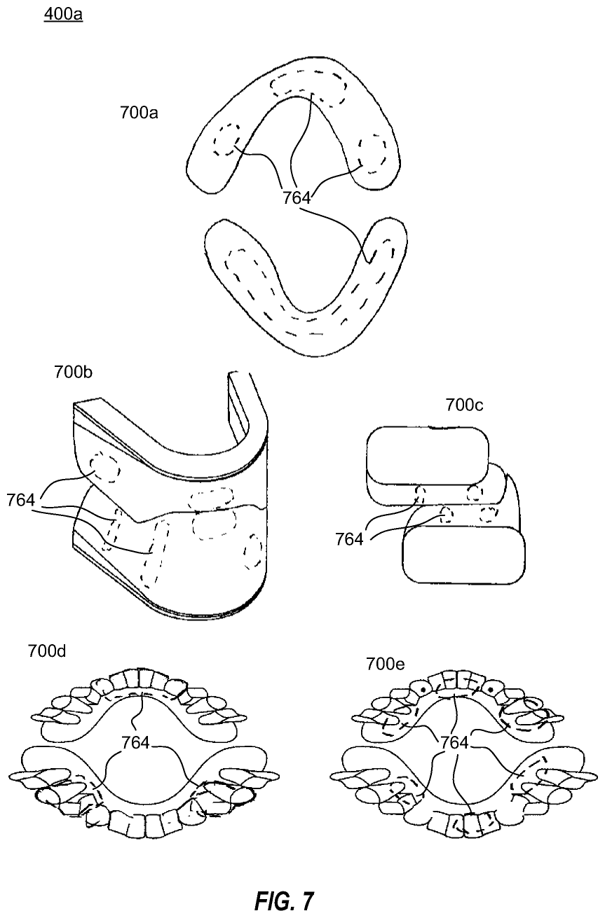

In addition, negative spaces, empty spaces, platforms, or attachment points may be useful in various combinations. FIG. 7 illustrate a combination of elements within dental appliances (700a-700c) and restorations (700d and 700e) according to an example embodiment. Negative or empty spaces 764 may be located completely within a dental solution 700a-700e and have no communication with the surface or may open onto the surface. Negative spaces 764 may have various sizes, shapes, and locations within dental solutions 700a-700e. In some cases, negative spaces 764 may be formed within prosthetic teeth, portions of prosthetic teeth, or across multiple prosthetic teeth. In some cases, these new features of a dental solution may provide for enhanced, alternative, or unorthodox treatments. As a non-limiting example, a researcher or clinician could place a device in the guidance package derived appliance or restoration that would vibrate when a patient bruxed at a certain intensity. The vibration could be used to condition the patient to stop bruxing in order to avoid the vibration, thereby preventing or limiting further tooth and tissue damage. Another non-limiting example would be the subsequent placement of magnets to encourage or discourage certain jaw position. Polarity of the magnets may be placed in a way to attract or repel.

Another nonlimiting example would be 3D x-ray (e.g., cone beam) correlated to a variety of sensors on the guidance package solution. Sensors may be included that may evaluate sound, movement, breathe analysis, pressure and any other variable to study, as non-limiting examples, function or dysfunction of a particular patient TMJ(s) and condylar movement, skeletal or dental disharmony in a particular patient, sleep apnea in a particular patient, and bruxism of a particular patient.

Sensor incorporation is unique with the guidance package system. A dental system according to some embodiments has the unique ability to create standardized appliances and/or restorations over a population group (e.g., a certain appliance/restorative design may be applied to many different patients customized based on their particular TMJ values). For the first time, different standardized appliance and restoration design theories may be studied in large population groups. Additionally, for an individual patient many different appliance designs may be easily generated and with the use of sensors may help find the best design.

As a non-limiting description, A guidance and stop pattern may be first internally reconfigured and then indexed to a different index position of mandible to maxilla other than maximum closure position. This alternative index position may be a position of the mandible in relation to the maxilla that exists within the scope of possible movements and stops of a particular guidance package solution.

Understanding that within any guidance package movement and stop solution dictated by the guidance package system, there is a continuum of 3D locations within that solution that the mandible/maxilla system (e.g., retentive piece system) could be indexed to the guidance pattern anywhere along this body of possible index positions. These are all potential alternative index positions from the maximum closure position.

It is often most practical to index the movement and stop profile to the retentive piece system in the maximum closure position. However, in some circumstances indexing the guidance profile in that position is not convenient. Accordingly, an operator may internally reconfigure the guidance package to be coincident with the designated different index position (other than maximum closure position) of the mandible to the maxilla. Then that manipulated guidance package may be indexed to that coincident position of mandible to maxilla into the virtual model at a virtual point designated from within the continuum of prescribed guidance package movement and stops.

Alternatively, the index position of mandible to maxilla may be applied first in the virtual articulator that may not be maximum closure position. At this point a guidance package solution may be constructed to provide appropriate guidance and stops that will allow indexing to retentive piece system in this alternative index position, or a guidance pattern may be internally manipulated to allow indexing into the retentive piece system at the alternative to maximum closure position of mandible to maxilla.

The guidance solutions may be virtually derived by a system using an understanding of the interference of surfaces as the mandible and maxilla are closed. Various mathematical transforms, morphs, or processes may be applied to create one or more potential dental solutions. The dental solutions may be derived with open or more of the additional features discussed herein.

In some cases, a plurality of dental solutions may be derived (e.g., parameterized) for one or more patients. Each dental device may be derived with a negative space for the inclusion of one or more sensors in order to study the effect of changes to design or patients on aspects of the dental solution (e.g., effectiveness).

Another non-limiting example could involve a hybrid orthodontic/guidance package appliance. In treating a patient who has a palate that is too narrow, a two-piece guidance package solution could include attachment points to provide expansive force similar to a rapid palatal expander or other type orthodontic appliances (see, e.g., FIG. 4A). Another example of an attachment point/platform/negative space built into a guidance package solution could include attachment to implant systems. In this example, the attachment point/platform/negative space is on the tissue side of the finished restoration (or appliance). This may be part of the parameterization of the retentive piece only or parameterization of retentive piece and guidance package. These attachment points may be formed in a way to attach directly to an implant permanently (i.e. a fixed hybrid, a denture type restoration fixed permanently to implants) or in a configuration that the appliance or restoration may be attached and removed by the patient (i.e. a removable implant supported denture). These negative spaces/attachment points may take on a form to accept an intermediary implant attachment mechanism that will accept an implant. The intermediary may be designed for permanent attachment to the implant or be detachable by the clinician or the patient (see, e.g., FIGS. 8A and 8B).

FIGS. 8A and 8B illustrate negative spaces, platforms, and attachment points according to certain example embodiments. As illustrated in FIGS. 8A and 8B, the appliances 800a and 800b includes maxillary retentive pieces 820a and 820b, respectively. Voids 825a and 825b are formed, respectively, in maxillary retentive pieces 820a and 820b. In FIG. 8A, void 825a provides an attachment point for implant 830a. In FIG. 8B, void 825b is formed to accept an attachment housing 832b, which then accepts implant 830b. Advantages of utilizing an intermediate housing may include: more options in attachment (e.g., attachment type and positioning); acceptance of different design platforms (e.g., brands) of implants; selectability of angulation of the implant; adjustability of the implant within the retentive piece (e.g., with an adjustable housing; inclusion of a permanent or temporary locking mechanism (e.g., to fix the retentive piece for/by a patient); and/or removable retentive pieces by the patient.

Negative or empty spaces may also be used for therapeutic medicaments to be dispensed under certain circumstances.

In some cases, the guidance package system may be used as an educational tool. For instance, the dental system has ability to readily convey principles of jaw movement for solutions to dental and medical problems for the first time in a virtual articulator. These principles of dentistry or new theories may be illustrated in regard to a specific patient or regarding population groups in a computer/virtual articulator environment for the first time. Different dental or guidance solutions may be applied using a system according to the example embodiment, and the changes to the movement profile or dental system of a particular patient or population groups may be analyzed in near real-time. Thus, an education tool may be provided using a system according to the present disclosure.

In some implementations, guidance package systems and/or appliances may be modified to address a plurality of distinct and/or overlapping issues. For example, in some embodiments, the guidance package system combined with retentive piece to create an appliance that can accomplish tooth movement and TMJ manipulation simultaneously. Such a system and appliance has increased utility from all the conventional orthodontic auxiliaries.

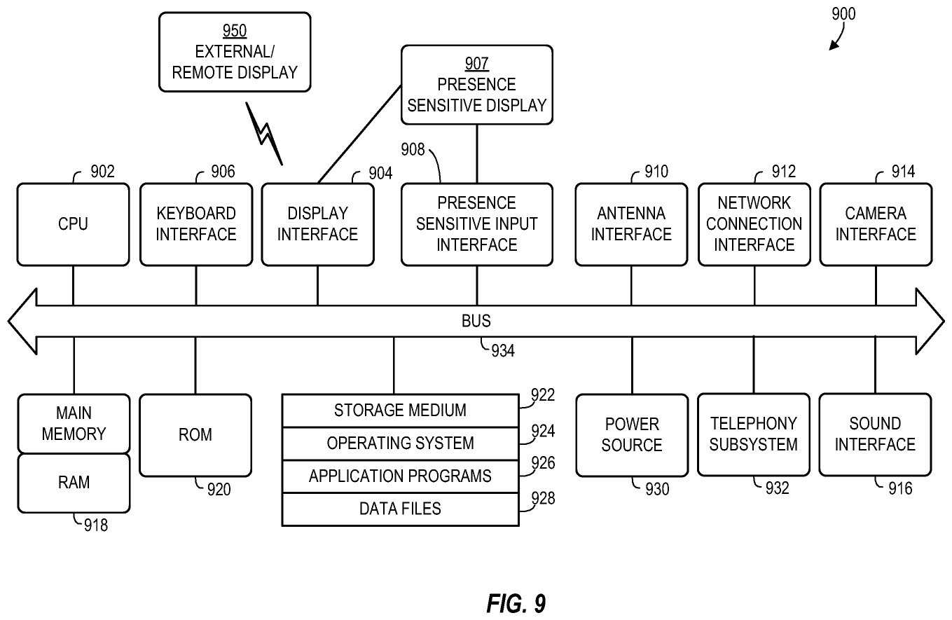

FIG. 9 illustrates an example computing device architecture that can implement one or more aspects of the present disclosure within a virtual articulator, a virtual package design system, and/or an appliance producer (e.g., a CAD/CAM system). One of ordinary skill will recognize that systems and devices having fewer, alternative, or additional components as that illustrated in FIG. 9 are within the scope of the present disclosure.

The computing device architecture 900 of FIG. 9 includes a central processing unit (CPU) 902, where computer instructions are processed, and a display interface 904 that acts as a communication interface and provides functions for rendering video, graphics, images, and texts on the display. In certain example implementations of the disclosed technology, the display interface 904 may be directly connected to a local display, such as a touch-screen display associated with a mobile computing device. In another example implementation, the display interface 904 may be configured for providing data, images, and other information for an external/remote display 950 that is not necessarily physically connected to the mobile computing device. For example, a desktop monitor may be used for mirroring graphics and other information that is presented on a mobile computing device. In certain example implementations, the display interface 904 may wirelessly communicate, for example, via a Wi-Fi channel or other available network connection interface 912 to the external/remote display 950.

In an example implementation, the network connection interface 912 may be configured as a communication interface and may provide functions for digital virtual assistant using voice, rendering video, graphics, images, text, other information, or any combination thereof on the display. In one example, a communication interface may include a microphone, camera, serial port, a parallel port, a general-purpose input and output (GPIO) port, a game port, a universal serial bus (USB), a micro-USB port, a high definition multimedia (HDMI) port, a video port, an audio port, a Bluetooth port, a near-field communication (NFC) port, another like communication interface, or any combination thereof. In one example, the display interface 904 may be operatively coupled to a local display, such as a touch-screen display associated with a mobile device or voice enabled device. In another example, the display interface 904 may be configured to provide video, graphics, images, text, other information, or any combination thereof for an external/remote display 950 that is not necessarily connected to the mobile computing device. In one example, a desktop monitor may be used for mirroring or extending graphical information that may be presented on a mobile device. In another example, the display interface 904 may wirelessly communicate, for example, via the network connection interface 912 such as a Wi-Fi transceiver to the external/remote display 950.

The computing device architecture 900 may include a keyboard interface 906 that provides a communication interface to a keyboard. In one example implementation, the computing device architecture 900 may include a presence sensitive input interface 908 for connecting to a presence sensitive display 907. According to certain example implementations of the disclosed technology, the presence sensitive input interface 908 may provide a communication interface to various devices such as a pointing device, a touch screen, a depth camera, microphone, etc. which may or may not be associated with a display.

The computing device architecture 900 may be configured to use an input device via one or more of input/output interfaces (for example, the keyboard interface 906, the display interface 904, the presence sensitive input interface 908, network connection interface 912, camera interface 914, sound interface 916, etc.) to allow a user to capture information into the computing device architecture 900. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 900 or may be a separate device. For example, the input device may be an accelerometer, a magnetometer, a digital camera, a microphone, and an optical sensor.

Example implementations of the computing device architecture 900 may include an antenna interface 910 that provides a communication interface to an antenna; a network connection interface 912 that provides a communication interface to a network. As mentioned above, the display interface 904 may be in communication with the network connection interface 912, for example, to provide information for display on a remote display that is not directly connected or attached to the system. In certain implementations, camera interface 914 acts as a communication interface and provides functions for capturing digital images from a camera. In certain implementations, a sound interface 916 is provided as a communication interface for converting sound into electrical signals using a microphone and for converting electrical signals into sound using a speaker. In certain implementations, a sound interface 916 is utilized to capture voice inputs for consumption by of other components connected to the BUS 934. According to example implementations, a random-access memory (RAM) 918 is provided, where computer instructions and data may be stored in a volatile memory device for processing by the CPU 902.

According to an example implementation, the computing device architecture 900 includes a read-only memory (ROM) 920 where invariant low-level system code or data for basic system functions such as basic input and output (I/O), startup, or reception of keystrokes from a keyboard are stored in a non-volatile memory device. According to an example implementation, the computing device architecture 900 includes a storage medium 922 or other suitable type of memory (e.g. such as RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 924, application programs 926 (including, for example, a web browser application, a widget or gadget engine, and or other applications, as necessary) and data files 928 are stored. According to an example implementation, the computing device architecture 900 includes a power source 930 that provides an appropriate alternating current (AC) or direct current (DC) to power components.

According to an example implementation, the computing device architecture 900 includes a telephony subsystem 932 that allows the computing device to transmit and receive sound over a telephone network. The constituent devices and the CPU 902 communicate with each other over a bus 934.

According to an example implementation, the CPU 902 has appropriate structure to be a computer processor. In one arrangement, the CPU 902 may include more than one processing unit. The RAM 918 interfaces with the computer BUS 934 to provide quick RAM storage to the CPU 902 during the execution of software programs such as the operating system application programs, and device drivers. More specifically, the CPU 902 loads computer-executable process steps from the storage medium 922 or other media into a field of the RAM 918 to execute software programs. Data may be stored in the RAM 918, where the data may be accessed by the computer CPU 902 during execution.

The storage medium 922 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 922, which may include a machine-readable storage medium.

According to one example implementation, the term computing device, as used herein, may be a CPU, or conceptualized as a CPU (for example, the CPU 902 of FIG. 9). In this example implementation, the computing device (CPU) may be coupled, connected, and/or in communication with one or more peripheral devices, such as display. In another example implementation, the term computing device, as used herein, may refer to a mobile computing device such as a smart phone, tablet computer, or smart watch. In this example implementation, the computing device may output content to its local display and/or speaker(s). In another example implementation, the computing device may output content to an external display device (e.g., over Wi-Fi) such as a TV or an external computing system.

In example implementations of the disclosed technology, a computing device may include any number of hardware and/or software applications that are executed to facilitate any of the operations. In example implementations, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be used to receive or collect data and/or user instructions from a wide variety of input devices. Received data may be processed by one or more computer processors as desired in various implementations of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

One skilled in the art would understand that these are merely examples, and a variety of modifications and alterations may be made within the scope of the present disclosure.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the invention is defined by the claims appended hereto.

What is claimed is:

1. A method of producing a retention piece having a therapeutic package configured to provide a desired therapeutic profile for a recipient, the method comprising:
obtaining data descriptive of mandibular and maxillary arches of the recipient;
specifying an appropriate guidance package based on the desired therapeutic profile for the recipient;
modeling a virtual representation of at least one retention piece configured to fit at least one of the mandibular and maxillary arches; and
generating data that can be used by a computer aided machining (CAM) process to create at least one physical retention piece comprising the desired therapeutic package configured to provide the desired therapeutic profile.

2. The method of claim 1, wherein the modeling the virtual representation of at least one retention piece comprises forming, within the virtual representation, one or more negative spaces.

3. The method of claim 2, wherein the one or more negative spaces is configured to produce a more shock absorbing area of the at least one retention piece.

4. The method of claim 2, further comprising creating the at least one physical retention piece based on the generated data, the at least one physical retention piece including, within the negative space, at least one from among an electrolytic gel, one or more sensors, a tracking device, a computer, and a microgenerator.

5. The method of claim 2, further comprising creating the at least one physical retention piece based on the generated data, the at least one physical retention piece including, within the negative space, an intermediary implant attachment mechanism that will accept an implant.

6. The method of claim 1, wherein the modeling the virtual representation of at least one retention piece comprises forming, within the virtual representation, a plurality of negative spaces connected by channels within the virtual representation.

7. The method of claim 1, wherein the modeling the virtual representation of at least one retention piece comprises forming, within the virtual representation, a plurality of attachment points on an external surface of the at least one retention piece.

8. The method of claim 7, further comprising creating the at least one physical retention piece based on the generated data, the at least one physical retention piece including, connected to the plurality of attachment points, at least one from among an elastic band, a guidance rod, a spacer, and an expansion screw.

9. The method of claim 1, wherein the therapeutic package comprises palatal suture manipulation, and the at least one retention piece comprises a rapid palatal expander.

10. The method of claim 1, further comprising modeling a plurality of virtual representations of at least one retention piece configured to fit at least one of the mandibular and maxillary arches and simulating a change of the mandibular and maxillary arches based on the plurality of virtual representations.

11. The method of claim 1, wherein modeling the virtual representation of the at least one retention piece comprises modeling the virtual representation based on at least one mathematical principle of aesthetics.

12. The method of claim 11, wherein the at least one mathematical principles of aesthetics comprises tooth size and shape in a continuum of parameterization.

13. The method of claim 1, wherein modeling the virtual representation of the at least one retention piece comprises modeling a manufactured surface on at least a portion of the at least one retention piece.

14. The method of claim 13, wherein the manufactured surface comprises at least one of a rough surface, a textured surface, and a meshed surface.

15. The method of claim 14, further comprising:
creating the at least one physical retention piece based on the generated data; and
performing a surface treatment to the manufactured surface.

16. A system for producing a retention piece having a therapeutic package configured to provide a desired therapeutic profile for a recipient, the system comprising:
at least one processor; and
at least one memory having stored thereon computer program code that, when executed by the at least one processor, controls the processor to:
obtain data descriptive of mandibular and maxillary arches of the recipient;
specify an appropriate guidance package based on the desired therapeutic profile for the recipient;
model a virtual representation of at least one retention piece configured to fit at least one of the mandibular and maxillary arches; and
generate data for a computer aided machining (CAM) process to create at least one physical retention piece comprising the desired therapeutic package configured to provide the desired therapeutic profile.

17. The system of claim 16, wherein the modeling the virtual representation of at least one retention piece comprises forming, within the virtual representation, one or more negative spaces.

18. The system of claim 17, wherein the one or more negative spaces is configured to produce a more shock absorbing area of the at least one retention piece.

19. The system of claim 17, wherein the computer program code, when executed by the at least one processor, further controls the processor to output instructions to create the at least one physical retention piece based on the generated data, the at least one physical retention piece including, within the negative space, at least one from among an electrolytic gel, one or more sensors, a tracking device, a computer, and a microgenerator.

20. The system of claim 16, wherein the modeling the virtual representation of at least one retention piece comprises forming, within the virtual representation, a plurality of negative spaces connected by channels within the virtual representation.
